(19) United States
(12) Patent Application Publication
(10) Pub. No.: US 2020/0179670 A1
(43) Pub. Date: Jun. 11, 2020

(54) RAPID-EXCHANGE SYSTEM AND METHOD

(71) Applicant: THE CLEVELAND CLINIC FOUNDATION, Cleveland, OH (US)

(72) Inventors: Karl West, Geneva, OH (US); Vikash Goel, McLean, VA (US); James Foster, Cleveland, OH (US)

(21) Appl. No.: 16/349,309

(22) PCT Filed: Nov. 10, 2017
(86) PCT No.: PCT/US2017/060995

§ 371 Date: May 13, 2019

Related U.S. Application Data
(60) Provisional application No. 62/420,606, filed on Nov. 11, 2016.

(51) Int. Cl. A61M 39/10; A61M 25/01

(52) U.S. Cl. CPC A61M 39/10; A61B 1/00124; A61M 25/01

(57) ABSTRACT

A connector for placing an elongate endovascular device and an electrical accessory item into electrical connection is described. A connector body with longitudinally separated proximal and distal body ends is electrically connected to the electrical accessory item. A connection cavity extends into, and partially through, the connector body from a cavity aperture in the distal body end. At least one electrical connector contact is located in the connection cavity. The electrical connector contact is configured for longitudinal alignment with, and electrical communication to, at least a conductive portion of the endovascular device selectively present in the connection cavity. A device grasper is operable to selectively maintain the conductive portion of the endovascular device longitudinally within the connection cavity. A method and system for using the connector are also disclosed.

### RELATED APPLICATION

This application claims priority from U.S. Provisional Application No. 62/420,606, filed 11 Nov. 2016, the subject matter of which is incorporated herein by reference in its entirety.

### TECHNICAL FIELD

This disclosure relates to a rapid exchange system and method and, more particularly, to a method, system, and apparatus for placing an elongate endovascular device and an electrical accessory item into electrical connection.

### BACKGROUND

Endovascular wires and catheters fitted with electronic devices are able to collect a wealth of information when traversed through the vascular system. Pressure, positional, flow, and oxygen sensors, for example, are being used currently with modified wires and catheters. The drawbacks to having sensors on the distal or working ends of these wires and catheters are the hardwired systems used to connect the sensors to the data collections system. That is, when a sensor is hardwired to a catheter or guidewire, the wires exiting the proximal end of that device greatly complicate the user's ability to, for example, exchange a different catheter over the sensor-equipped guidewire while leaving the guidewire in place in a patient's body.

### SUMMARY

In an example, a connector for placing an elongate endovascular device and an electrical accessory item into electrical connection is described. A connector body with longitudinally separated proximal and distal body ends is electrically connected to the electrical accessory item. A connection cavity extends into, and partially through, the connector body from a cavity aperture in the distal body end. At least one electrical connector contact is located in the connection cavity. The electrical connector contact is configured for longitudinal alignment with, and electrical communication to, at least a conductive portion of the endovascular device selectively present in the connection cavity. A device grasper is operable to selectively maintain the conductive portion of the endovascular device longitudinally within the connection cavity.

In an example, a rapid-exchange system for endovascular use is described. An elongate endovascular device includes a distal working end and a proximal connection end. The proximal connection end of the endovascular device includes a conductive portion with at least one electrical device contact. The distal working end includes a device feature in electrical communication with the at least one electrical device contact. An electrical accessory item is provided for selective electrical connection with the device feature. A connector is provided for placing the elongate endovascular device and the electrical accessory item into electrical connection. The connector includes a connector body with longitudinally separated proximal and distal body ends. A connection cavity extends into, and partially through, the connector body from a cavity aperture in the distal body end. The connection cavity is configured to selectively accept the conductive portion of the endovascular device therein. At least one electrical contact is located in the connection cavity. The electrical connector contact is configured for longitudinal alignment with, and electrical connection to, a corresponding one of the at least one electrical device contact. A device grasper is operable to selectively maintain the conductive portion of the endovascular device longitudinally within the connection cavity. The device grasper is selectively actuated, with the conductive portion of the endovascular device located in the connection cavity, to maintain the electrical connection between the electrical connector contact and the electrical device contact.

In an example, a method of placing an elongate endovascular device and an electrical accessory item into electrical connection is described. A connector is provided. The connector includes a connector body with longitudinally separated proximal and distal body ends. A connection cavity extends into, and partially through, the connector body from a cavity aperture in the distal body end. At least one electrical connector contact is located in the connection cavity. The connector includes a selectively actuable device grasper. The electrical accessory item is placed into electrical contact with the electrical connector contact. A proximal connection end, including a conductive portion having at least one electrical device contact, of the elongate endovascular device is placed into the connection cavity. The electrical connector contact is longitudinally aligned with the conductive portion of the elongate endovascular device. The electrical connector contact and the electrical device contact are placed into electrical communication while at least the conductive portion of the elongate endovascular device is located in the connection cavity. The device grasper is actuated to selectively maintain the conductive portion of the endovascular device longitudinally within the connection cavity.

### BRIEF DESCRIPTION OF THE DRAWINGS

For a better understanding, reference may be made to the accompanying drawings, in which:

FIG. 1 is a schematic partial side view of an example of the invention;

FIG. 2 is a schematic partial sectional side view of a first example configuration of the example of FIG. 1;

FIG. 3 is a schematic perspective view of a second example configuration of the example of FIG. 1;

FIG. 4 is a schematic partial sectional side view of a third example configuration of the example of FIG. 1;

FIG. 5 is a front view of the third example configuration of FIG. 4;

FIG. 6 is a detail view of area “6” of FIG. 4;

FIG. 7 is a partial sectional side view of a component of the third example configuration of FIG. 4;

FIG. 8 is a schematic partial side view of a component of the example of FIG. 1;

FIG. 9 is a flowchart of an example sequence of operation of the example of FIG. 1;

FIG. 10 is a partial side view of the example of FIG. 1;

FIG. 11 is a partial side view of a stage of the example sequence of operation of FIG. 9;

FIG. 12 is a partial front perspective view similar to FIG. 11;

FIG. 13 is a partial side view of a stage of the example sequence of operation of FIG. 9;

FIG. 14 is a partial front perspective view similar to FIG. 13;

FIG. 15 is a partial side view of a stage of the example sequence of operation of FIG. 9; and

FIG. 16 is a partial side view of a stage of the example sequence of operation of FIG. 9.

### DETAILED DESCRIPTION

The invention comprises, consists of, or consists essentially of the following features, in any combination.

In summary, examples of the present invention provide a rapid-exchange system for endovascular use. The device grasper 226 can selectively maintain or hold the elongate endovascular device 102 at a predetermined longitudinal and rotational position within the connection cavity 112 of the connector 100. For example, the device grasper 226 can be magnetic, detent, frictional, mechanical, and/or any other type of mechanism operative to help align the electrical connector contacts 116 and electrical device contacts 822 to enable transmission of power and/or data. The electrical transmission of power and/or data between the connector contacts 116 and electrical device contacts 822 may be through a contact or non-contact connection.

At least one electrical contact 116 is located in the connection cavity 112. The electrical connector contact 116 is configured for longitudinal alignment with, and electrical connection to, a corresponding one of the at least one electrical device contact(s) 822. The device grasper 226 is selectively actuated, with the conductive portion of the elongate endovascular device 102 located in the connection cavity 112, to maintain the electrical connection between the electrical connector contact 116 and the electrical device contact 822. The connector 100 can be easily disconnected from the elongate endovascular device 102 by simply pulling the two devices apart (optionally after briefly manipulating the device grasper 226), therefore allowing the elongate endovascular device 102 to be exchanged and the same, or a different, elongate endovascular device 102 to be quickly reconnected to the electrical accessory item 104.

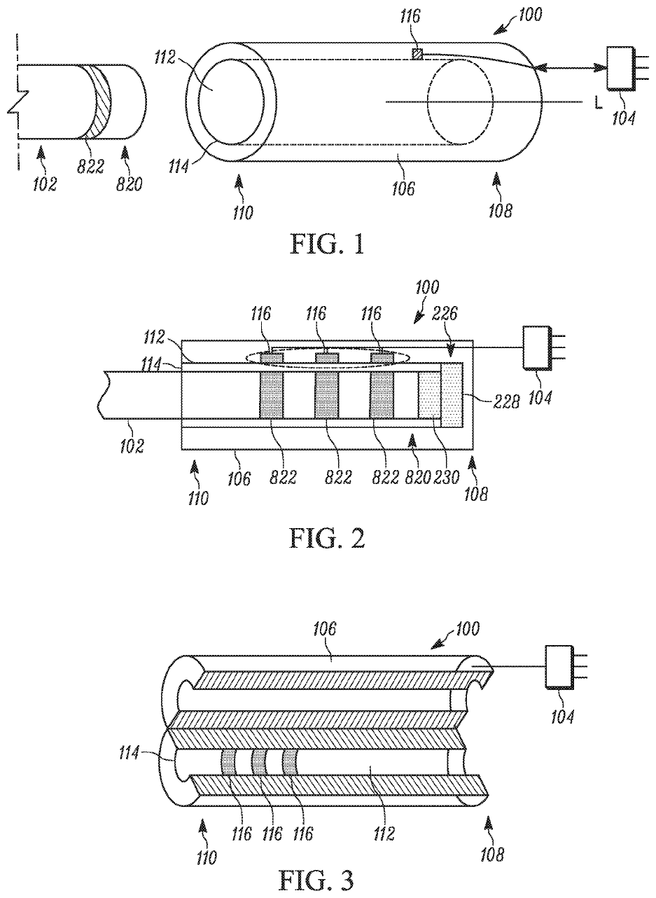

FIG. 1 depicts a connector 100 for placing an elongate endovascular device 102 and at least one electrical accessory item 104 into electrical connection and/or mechanical connection. The electrical connection(s) between the elongate endovascular device 102 and the at least one electrical accessory item 104 could be of the wired and/or wireless type, though is shown schematically in FIG. 1 as being wired. In other examples, however, the electrical accessory item 104 and the connector 100 can include a wireless (e.g., inductive or capacitive) link therebetween for wireless data and/or power transmission. The communication link between the electrical accessory item 104 and the connector 100 can, in any configuration provide for communication of data and/or power between the connector 100 and the electrical accessory item 104, and may include unidirectional or bidirectional communications.

The elongate endovascular device 102 could be of any desired type such as, but not limited to, any combination of a catheter, a guidewire, an endoscope, a dilator, a stylet, an electrocautery device, and/or the like that includes one or more transducers that send and/or receive signals to/from the electrical accessory item 104. The electrical accessory item 104 could be of any desired type such as, but not limited to, any combination of a power source, a computer, a data collection system, an imaging device, an electrical signal monitoring and/or display device, and/or the like. In one example, the electrical accessory item 104 is an electromagnetic tracking system, such as the type of tracking system that is commercially available from Northern Digital, Inc., of Ontario, Canada. Any desired number of intermediate plugs, extension lines, connections, or the like may be provided in-line electrically between the connector 100 and the electrical accessory item 104, as desired.

The connector 100 includes a connector body 106 with longitudinally separated proximal and distal body ends 108 and 110, respectively. The connector body 106 is electrically, and optionally mechanically, connected to the electrical accessory item 104, as shown schematically in FIG. 1. As illustrated in the Figures, the connector body 106 can have a cylindrical form. However, the connector body 106 can be of any desired shape, including, but not limited to, rectilinear, curvilinear, ergonomically profiled. It is contemplated that, in some use environments, the connector 100 could be configured to also serve as a torquer for the elongate endovascular device.

A connection cavity 112 extends into, and partially through, the connector body 106 from a cavity aperture 114 in the distal body end 110. It is contemplated that, for some example configurations of the connector 100, the connection cavity 112 will be a blind-ended hole to facilitate longitudinal positioning of the elongate endovascular device 102 as described below.

In some examples, the connector body 106 includes at least one electrical connector contact 116 that is located in the connection cavity 112. The electrical connector contact 116 is configured for longitudinal alignment with, and electrical connection to, at least a conductive portion of the endovascular device 102 when that elongate endovascular device is selectively present in the connection cavity 112, as will be discussed further below. The connector body 106 places the electrical accessory system 104 into electrical communication with the at least one electrical connector contact 116, such as via the internal wires shown schematically in the Figures. Electrical communication is used herein at least in part to reference an electrical connecting including contact or non-contact (wireless) connections that provide for communication or power and/or data transfer. In other examples, the connector body 106 includes a wireless connection (e.g., an inductive link or the like) that provides for the electrical communication of the signal (or signals) between the endovascular device 102 and the electrical accessory device 104,

With reference now to FIG. 8, an elongate endovascular device 102 includes a distal working end 818 and a proximal connection end 820. The proximal connection end 820 of the endovascular device 102 includes a conductive portion with at least one electrical device contact 822 (four shown in FIG. 8). The distal working end includes a device feature 824 in electrical communication (e.g., via wires internal to the elongate endovascular device 102) with the at least one electrical device contact 822.

The device feature 824 may be any desired type or combination thereof which uses electrical power and/or involves the use of transmitted data, including, but not limited to, one or more of: an imaging or other visualization tool, an electrocautery tool, a pressure sensor, a position/location/orientation sensor, a temperature sensor, a flow sensor, a transducer of any type, an oxygen sensor, or the like.

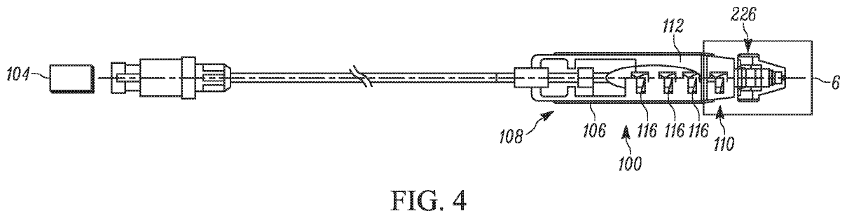

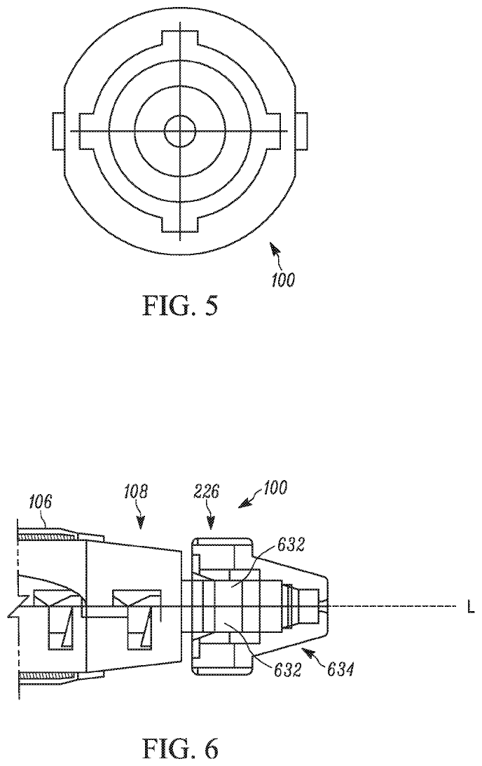

Four electrical device contacts 822 are illustrated in FIG. 8, by way of example, but any desired number of electrical device contacts 822 may be provided. The number and/or location(s) of the electrical device contacts 822 may be adapted to correspond to respective device features 824 and the number of signals being communicated relative to the device 102. The connector 100 can have a number/location(s) of electrical connector contacts 116 which correspond to the electrical device contacts 822 for at least one particular elongate endovascular device 102. The electrical device contacts 822, and/or electrical connector contacts 116 may be at least partially made from a suitable conductive material, and may be configured in any desired manner. For example, the electrical device contacts 822 and/or electrical connector contacts 116 may be a “band” or ring about their respective elongate endovascular device 102 or connector 100 surface, to allow for some leeway in radial (i.e., about the longitudinal axis L) positioning relative to one another. As another example, one or more of the electrical connector contacts 116 may be spring-biased into selective electrical connection with a corresponding electrical device contact 822, as shown in the examples of FIGS. 4-7.

Any or all of the electrical connections made using the connector 100 (e.g., between the electrical accessory item 104 and the electrical connector contact 116, the electrical connector contact 116 and the electrical device contact 822, and/or the electrical device contact 822 and the device feature 824) may be one or both of a data-transmitting electrical connection and a power-transmitting electrical connection.

The electrical connector contacts 116 can each communicatively couple (e.g., via contact or non-contact) with the mating electrical device contacts 822 which are in electrical (e.g., power and/or data transmission and/or receiving contact) connection with the device features 824. In some cases, depending on the device feature 824 function(s) and the way signals are communicated (e.g., differential signals, bipolar signals or the like), more than one electrical device contact 822 may be used for communicating information collected by a given sensor or provided to a given transducer.

In short, then, the electrical accessory item 104 is provided for selective electrical connection with one or more device feature(s) 824 of the elongate endovascular device 102, and the connector 100 places the elongate endovascular device 102 and the electrical accessory item 104 into one or both of electrical and mechanical connection with one another. The elongate endovascular device 102 can be, for example, a guide wire, a catheter, or the like, fitted with electronic sensors, power application tools, or any other type of desired device feature(s) 824.

Some existing types of elongate endovascular devices 102 require direct hardwired connection between the device features 824 and the corresponding electrical accessory item(s) 104. The hardwired connection usually involves wires extending from the proximal end of the elongate endovascular device 102, making it difficult, if not impossible, to exchange the elongate endovascular device 102 or other associated devices within the patient's vasculature “over the wire” without complicated modifications, such as completely disconnecting the electrical accessory item 104 system. The connector 100 disclosed herein eliminates this lengthy and unwieldy directly hardwired connection between the device features 824 and the electrical accessory item 104 which was heretofore made via wires extending from the distal end of the elongate endovascular device 102. Additionally, different elongate endovascular devices 102 can be swapped into and/or out of the connector 100, in examples of this disclosure, without changing the connection between the connector 100 and the electrical accessory item 104. Thus, the connector 100 allows, for example, for data collected by one or more sensors (as device features 824) to be transmitted to a data collection system (as an electrical accessory item 104), while still allowing for quick exchange of elongate endovascular devices 102 within the patient's body to a different device without requiring reconnecting a device to the electrical accessory item 104.

Turning back to FIGS. 1-4, the connector 100 can interface with the elongate endovascular device 102 in any desired manner. For example, and as shown in FIGS. 1-2, the connector 100 could slide longitudinally (i.e., along longitudinal axis “L”, as shown in FIG. 1) with respect to the elongate endovascular device 102, thus placing at least a conductive portion (i.e., a portion including at least one electrical device contact 822) of the elongate endovascular device 102 into the connection cavity 112. It is contemplated that, some examples, the electrical connector contact(s) 116 and the electrical device contact(s) 822 would be longitudinally aligned—for example, by abutment of the proximal connection end 820 of the elongate endovascular device 102 against the blind end of the connection cavity 112—when the connector 100 is properly “seated” on the elongate endovascular device 102. As another example, and as shown in FIG. 3, the connector 100 can have a “clamshell”-type design to facilitate positioning the proximal connection end 820 of the elongate endovascular device 102 within the connector 100. For instance, the two hinged-apart portions of the clamshell connector 100 housing can be urged together around the proximal connection end 820 of the elongate endovascular device 102 and clipped shut (e.g., via a clip or fastener, not shown) to “lock” the electrical connector contact(s) 116 into position with respect to the electrical device contact(s) 822. For either of the configurations of FIGS. 1-3, the connection cavity 112 and proximal connection end 820 could be arranged to stay in their relative longitudinal position without further fastening structure, such as via a relative frictional or interference fit.

More broadly stated, a device grasper 226, of any desired locking or retaining mechanism type, may be operable to selectively maintain the conductive portion of the elongate endovascular device 102 longitudinally within the connection cavity 112. For example, as shown in the example of at least FIGS. 1 and 3, the device grasper 226 feature may be fulfilled, as previously mentioned, by a frictional or interference fit between components of the connector 100 and elongate endovascular device 102. Optionally, a detent of another type (e.g., bayonet-plug, adhesive, captured-ball, or any other desired structure) could serve as a device grasper 226, alone or in combination with another type of device grasper 226.

As another example, and as shown in the example of FIG. 2, the device grasper 226, which may be a magnet 228 and/or another type of magnetically sensitive material, interacts magnetically with a magnetically-sensitive feature 230 of the elongate endovascular device 102 to maintain the conductive portion of the endovascular device 102 longitudinally within the connection cavity 112. For example, a suitably strong magnet 228 could be located at the blind end of the connection cavity 112, with either another, attractively-arranged magnet or a magnetically-attracted material serving as a “cap” on the proximal-most end of the elongate endovascular device 102. As a result, when these two magnetic features 228 and 230 are brought into close proximity, they attract together strongly enough to maintain the elongate endovascular device 102 longitudinally within the connection cavity 112 sufficiently to maintain the electrical communication of signals (e.g., providing connections for power and/or data) between the transducer at the distal end of the device 102 and the electrical accessory item 104. The magnetic features 228 and 230 thus afford rotation for a particular use environment, while still providing the desired rapid-exchange or quick-connect features to the connector 100 system.

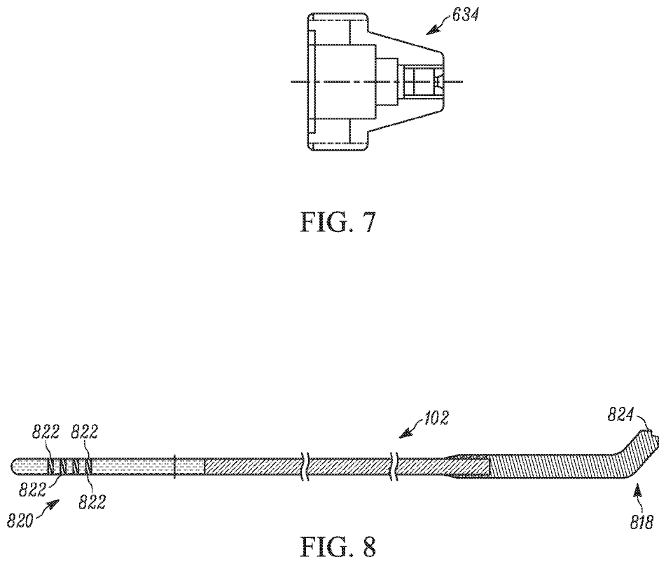

With reference now to FIGS. 4-7, the device grasper 226, in another example, may include a plurality of collet fingers 632 and a connector collar 634 (shown alone in FIG. 7). The connector collar 634 may selectively interact with the collet fingers 632 to urge the collet fingers 632 mutually radially inward (i.e., toward longitudinal axis “L”) to selectively grasp the elongate endovascular device 102, when inserted at least partially into the connection cavity 112. For example, the connector collar 634 may threadably engage with the collet fingers 632 to urge the collet fingers 632 mutually radially inward, in a “chuck”-type manner, to selectively grasp the elongate endovascular device 102.

To that end, a plurality of collet fingers 632 (which might simply be longitudinally slit portions of the connector body 106 surrounding the cavity aperture 114) are provided at any suitable location in which they can be used to pinch or hold the elongate endovascular device 102. For example, the collet fingers 632 and connector collar 634 may be located at the distal body end 110, as shown in the Figures.

The device grasper 226 may maintains a predetermined rotational position of the endovascular device 102 about the longitudinal axis L while the conductive portion of the endovascular device 102 is maintained longitudinally within the connection cavity 112 (e.g., providing six degrees of freedom fixation). This may be helpful, for example, if it is desired to use of the connector 100 as a torquer or as an indicator of a radial position of a device feature 824 at the distal working end 818 of the elongate endovascular device 102. The collet-type device grasper 226 of FIGS. 4-7 may be helpful, if firmly tightened, in providing such a “rotationally-fixed” arrangement.

Conversely, such as in other use environments, the device grasper 226 may permit a predetermined amount of rotation (anywhere from a few degrees to free-spinning) of the endovascular device 102 about the longitudinal axis L while the conductive portion of the endovascular device 102 is maintained longitudinally within the connection cavity 112 (e.g., providing five degrees of freedom fixation—allowing for rotation). The device grasper 226 may thus allow the elongate endovascular device 102 to rotate within the connector 100 without the risk of detachment from the electrical connector contact(s) 116 and resultant loss of electrical connection between the connector 100, the elongate endovascular device 102, and the electrical accessory item 104. The magnetic device grasper 226 of FIG. 2 may be helpful in providing such a “rotation-permitted” arrangement.

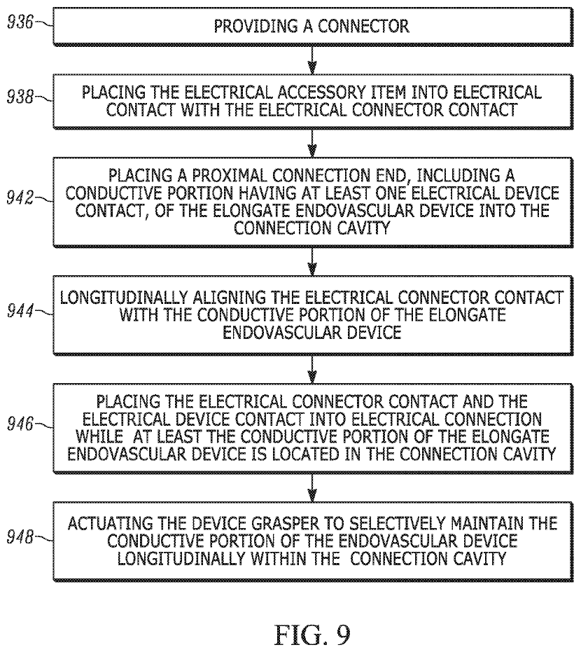

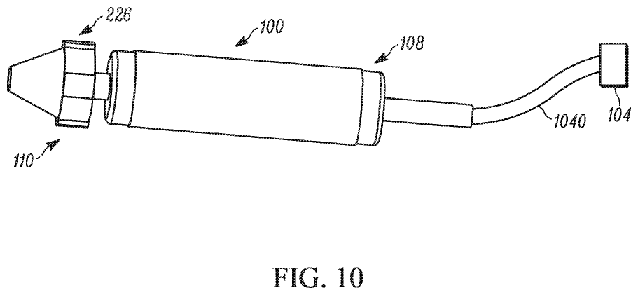

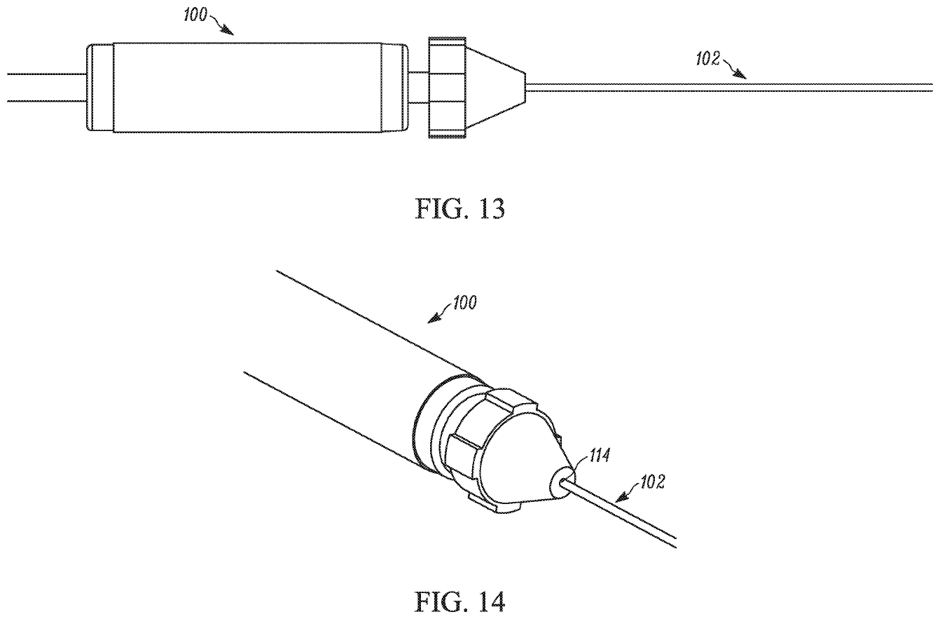

A method of placing an elongate endovascular device 102 and an electrical accessory item 104 into electrical connection is set forth schematically in the flowchart of FIG. 9, and depicted visually in the sequence of FIGS. 10-16. While, for purposes of simplicity of explanation, the example method is shown and described as executing serially, the method is not limited by the illustrated order, as some actions could, in other examples, occur in different orders and/or concurrently with other actions from that shown and described herein. Moreover, not all illustrated and described features may be required to implement a method.

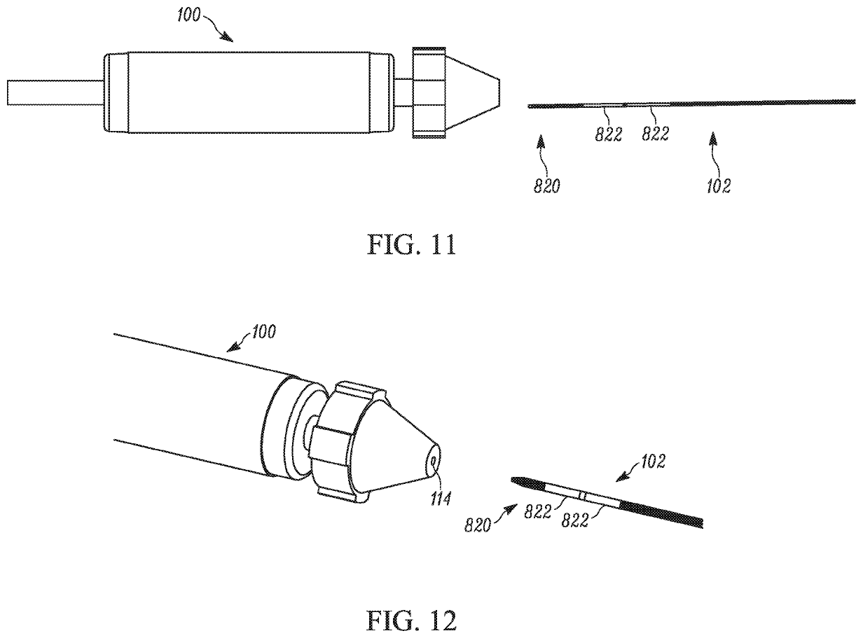

As shown in the first action block 936 of FIG. 9, a connector 100 is provided. The connector 100, an example of which is shown in FIG. 10, includes a connector body 106 with longitudinally separated proximal and distal body ends 108 and 110, respectively. A connection cavity 112 extends into, and partially through, the connector body 106 from a cavity aperture 114 in the distal body end 110. Though the connection cavity 112 is depicted herein as being a closed or blind-ended void, to facilitate longitudinal positioning of the elongate endovascular device 102, it is also contemplated that the connection cavity 112 could extend entirely through the connector body 106 and the longitudinal positioning assistance could be provided in a different manner. At least one electrical connector contact 116 is located in the connection cavity 112. The connector also includes a selectively actuable device grasper 226. The connector 100 and elongate endovascular device 102 are shown separately, though aligned for connection, in FIGS. 11-12.

In second action block 938, the electrical accessory item 104 (shown schematically in FIG. 10) is placed into electrical contact with the electrical connector contact 116. This can be accomplished, for example, via any suitable wired or wireless connection between the electrical accessory item 104 and the electrical connector contact 116, either directly or through one or more intermediate members. As an example, the connection could be made through wiring internal to the connector 100 and/or a connecting wire 1040 (shown in FIG. 10) which is hardwired into, and/or attached via plug to, the electrical accessory item 104 for power and/or data transmission.

In third action block 942, a proximal connection end 820, including a conductive portion having at least one electrical device contact 822, of the elongate endovascular device 102 is placed into the connection cavity 112, such as via the longitudinally sliding insertion into the cavity aperture 114 which has been accomplished between the views in FIGS. 11/12 and 13/14. Proceeding to fourth action block 944, the electrical connector contact 116 of the connector 100 is longitudinally aligned with the conductive portion (which includes at least one electrical device contact 822) of the elongate endovascular device 102. For example, when the connector 100 is dimensioned for such, the elongate endovascular device 102 could be inserted until it “bottoms out” at the blind end of the connection cavity 112 or enters into a mechanical detent device, to prevent further insertion past a desired longitudinal position.

Through any desired method (such as, but not limited to, limiting longitudinal insertion of the proximal connection end 820 into the connection cavity 112), the electrical connector contact 116 and the electrical device contact 822 are placed into electrical connection while at least the conductive portion of the elongate endovascular device 102 is located in the connection cavity 112, in fifth action block 946. As previously discussed, placing the electrical connector contact 116 and the electrical device contact 822 into electrical connection may include spring-biasing the electrical connector contact 116 into electrical connection with the electrical device contact 822

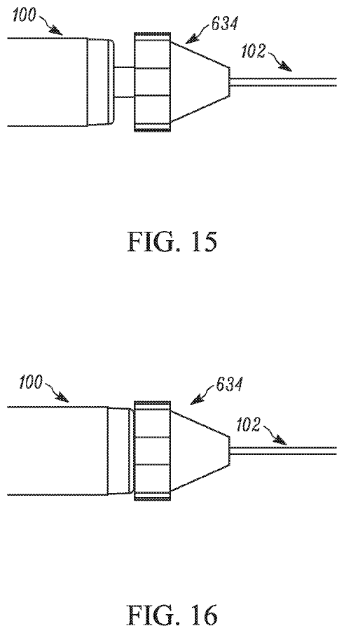

In sixth action block 948, the device grasper 226 selectively maintains the conductive portion of the endovascular device 102 longitudinally within the connection cavity 112. For example, as shown in the sequence of FIGS. 15-16, when the device grasper 226 includes a plurality of collet fingers 632 and a connector collar 634, then actuating the device grasper 226 may include actuating the connector collar 634 (e.g., by threadably engaging another portion of the connector 100) to selectively interact with the collet fingers 632 to urge the collet fingers 632 mutually radially inward to selectively grasp the elongate endovascular device 102. As another example, actuation of the device grasper 226 may include bringing magnetic features 228 and 230 into sufficiently close contact for a desired attraction therebetween to occur, engaging a detent mechanism, or any other desired actuation for a particular type of device grasper.

Regardless of when or how the device grasper 226 is actuated, the method may also include transmitting data and/or power through the electrical connections between the electrical accessory item 104 and the electrical connector contact 116, and/or between the electrical connector contact 116 and a corresponding electrical device contact 822.

The elongate endovascular device 102 may be used as desired while connected to the electrical accessory item 104 via the connector 100. As desired by the user, the device grasper 226 can then be de-actuated and the elongate endovascular device 102 removed from the connector 100. This elongate endovascular device could then be connected to a second electrical accessory item 104 via the same, or a different, connector 100. A second elongate endovascular device 102 can be provided and connected to the electrical accessory item 104 via the connector 100, similarly. Any desired number, combination, and arrangement of elongate endovascular device 102, electrical accessory item 104, and/or connector 100 could be provided to make the described electrical connections as desired for a particular use environment.

It is contemplated that the connector 100 could include one or more “patterns” of variously arranged electrical connector contacts 116, to allow for differing electrical connections to different elongate endovascular devices 102. For example, one set of electrical connector contacts 116, associated with a first elongate endovascular device 102a (e.g., a flow sensor), could be located radially and/or longitudinally offset, and/or have a different location pattern, from a second set of electrical connector contacts 116, associated with a second elongate endovascular device 102b (e.g., an electrocautery knife). In this manner, the connector 100 could be used with a number of different data and/or power transfer electrical accessory items 104 while avoiding placing an electrically unsuitable elongate endovascular device 102 into electrical contact with a particular electrical accessory item 104. It is also contemplated that in some examples, the connector 100 can have embedded electronics to automatically identify different elongate endovascular devices 102 and/or electrical device contacts 822, such as physically (via a pattern or arrangement of the electrical device contacts 822), electrically (via a signal transmitted from the device features 824), or in any other desired manner.

Unless defined otherwise, all technical and scientific terms used herein have the same meaning as is commonly understood by one of skill in the art to which the present disclosure pertains.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

While examples of this disclosure have been particularly shown and described herein, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one example or configuration could be provided, singly or in combination with other structures or features, to any other example or configuration, as it would be impractical to describe each of the examples and configurations discussed herein as having all of the options discussed with respect to all of the other examples and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

Having described the invention, we claim:

1-20. (canceled)

21. A connector for placing an elongate endovascular device and an electrical accessory item into electrical connection, the connector comprising:
a connector body with longitudinally separated proximal and distal body ends, the connector body being electrically connected to the electrical accessory item, with a connection cavity extending into, and partially through, the connector body from a cavity aperture in the distal body end;
at least one electrical connector contact located in the connection cavity, the electrical connector contact configured for longitudinal alignment with, and electrical communication to, at least a conductive portion of the endovascular device selectively present in the connection cavity; and
a device grasper operable to selectively maintain the conductive portion of the endovascular device longitudinally within the connection cavity.

22. The connector of claim 21, wherein at least one electrical connection is a data-transmitting electrical connection.

23. The connector of claim 21, wherein at least one electrical connection is a power-transmitting electrical connection.

24. The connector of claim 21, wherein the device grasper maintains a predetermined rotational position of the endovascular device about the longitudinal axis while the conductive portion of the endovascular device is maintained longitudinally within the connection cavity.

25. The connector of claim 21, wherein the device grasper permits a predetermined amount of rotation of the endovascular device about the longitudinal axis while the conductive portion of the endovascular device is maintained longitudinally within the connection cavity.

26. The connector of claim 21, wherein the device grasper interacts magnetically with a magnetically-sensitive feature of the endovascular device to maintain the conductive portion of the endovascular device longitudinally within the connection cavity.

27. The connector of claim 21, wherein the device grasper includes a plurality of collet fingers and a connector collar, the connector collar selectively interacting with the collet fingers to urge the collet fingers mutually radially inward to selectively grasp the endovascular device.

28. The connector of claim 27, wherein the collet fingers and connector collar are located at the distal body end.

29. The connector of claim 27, wherein the connector collar threadably engages with the collet fingers to urge the collet fingers mutually radially inward to selectively grasp the endovascular device.

30. The connector of claim 21, wherein the connector body places the electrical accessory item into electrical connection with the at least one electrical connector contact.

31. A rapid-exchange system for endovascular use, the system comprising:
an elongate endovascular device including a distal working end and a proximal connection end, the proximal connection end of the endovascular device including a conductive portion with at least one electrical device contact, and the distal working end including a device feature in electrical communication with the at least one electrical device contact;
an electrical accessory item for selective electrical connection with the device feature; and
a connector for placing the elongate endovascular device and the electrical accessory item into electrical connection, the connector including
a connector body with longitudinally separated proximal and distal body ends, with a connection cavity extending into, and partially through, the connector body from a cavity aperture in the distal body end, the connection cavity being configured to selectively accept the conductive portion of the endovascular device therein,
at least one electrical contact located in the connection cavity, the electrical connector contact configured for longitudinal alignment with, and electrical connection to, a corresponding one of the at least one electrical device contact, and
a device grasper operable to selectively maintain the conductive portion of the endovascular device longitudinally within the connection cavity;
wherein the device grasper is selectively actuated, with the conductive portion of the endovascular device located in the connection cavity, to maintain the electrical connection between the electrical connector contact and the electrical device contact.

32. The system of claim 31, wherein the electrical connector contact is spring-biased into selective electrical connection with the electrical device contact.

33. The system of claim 31, wherein the device grasper maintains a predetermined rotational position of the endovascular device about the longitudinal axis while the conductive portion of the endovascular device is maintained longitudinally within the connection cavity.

34. The system of claim 31, wherein the device grasper permits a predetermined amount of rotation of the endovascular device about the longitudinal axis while the conductive portion of the endovascular device is maintained longitudinally within the connection cavity.

35. The system of claim 31, wherein the device grasper includes a plurality of collet fingers and a connector collar, the connector collar selectively interacting with the collet fingers to urge the collet fingers mutually radially inward to selectively grasp the endovascular device when the endovascular device is at least partially located within the connection cavity.

36. A method of placing an elongate endovascular device and an electrical accessory item into electrical connection, the method comprising:
providing a connector, the connector including
a connector body with longitudinally separated proximal and distal body ends, with a connection cavity extending into, and partially through, the connector body from a cavity aperture in the distal body end,
at least one electrical connector contact located in the connection cavity, and
a selectively actuable device grasper;
placing the electrical accessory item into electrical contact with the electrical connector contact;
placing a proximal connection end, including a conductive portion having at least one electrical device contact, of the elongate endovascular device into the connection cavity;
longitudinally aligning the electrical connector contact with the conductive portion of the elongate endovascular device;
placing the electrical connector contact and the electrical device contact into electrical communication while at least the conductive portion of the elongate endovascular device is located in the connection cavity; and
actuating the device grasper to selectively maintain the conductive portion of the endovascular device longitudinally within the connection cavity.

37. The method of claim 36, including transmitting data through the electrical connections between the electrical accessory item and the electrical connector contact and between the electrical connector contact and the electrical device contact.

38. The method of claim 36, including transmitting power through the electrical connections between the electrical accessory item and the electrical connector contact and between the electrical connector contact and the electrical device contact.

39. The method of claim 36, wherein placing the electrical connector contact and the electrical device contact into electrical connection includes spring-biasing the electrical connector contact into electrical connection with the electrical device contact.

40. The method of claim 36, wherein the device grasper includes a plurality of collet fingers and a connector collar and wherein actuating the device grasper includes actuating the connector collar to selectively interact with the collet fingers to urge the collet fingers mutually radially inward to selectively grasp the endovascular device.
